(19) United States
(12) Patent Application Publication
(10) Pub. No.: US 2006/0139753 A1
(43) Pub. Date: Jun. 29, 2006

(54) Microscope drape coupling system and method

(76) Inventors: Gary L. Moses, Richmond, VA (US); Robin Charles Humble, Newcastle under Lyme (GB)

Correspondence Address: BAKER BOTTS L.L.P., 2001 ROSS AVENUE, SUITE 600, DALLAS, TX, 75201-2980, US

(21) Appl. No.: 11/285,785

(22) Filed: Nov. 21, 2005

Related U.S. Application Data
(63) Continuation of application No. 10/698,745, filed on Oct. 31, 2003.

(51) Int. Cl. A61B 19/08

(52) U.S. Cl. 359/510; 359/507

(57) ABSTRACT

According to one embodiment of the invention, a microscope drape coupling system includes a first adapter configured to couple to a first objective lens barrel of a first microscope, a housing configured to rotatably couple to the first adapter, and a substantially flat transparent protective lens coupled within the housing such that a geometric normal to the transparent protective lens forms an angle with respect to an optical axis of a first objective lens housed within the first objective lens barrel when the housing is coupled to the first objective lens barrel.

### TECHNICAL FIELD OF THE INVENTION

The present invention relates generally to the field of medical drapes and, more particularly, to a microscope drape coupling system and method.

### BACKGROUND OF THE INVENTION

To minimize the risk of infection to surgical patients in an operating room or to protect medical equipment from a surgical field during surgery, drapes are often utilized. Drapes may be placed over a patient and/or medical equipment to form a sterile barrier, keeping any microorganisms and contaminants that may cause infections from migrating to and from exposed tissue and open wounds. For example, bodily fluids during surgery may settle on medical equipment, which then become contaminated and hazardous to those persons who must work with the equipment. Instead, the bodily fluids will ultimately settle on the drapes and not on the draped medical equipment.

The advancement of medical procedures has correspondingly created a demand for more advanced medical equipment. For example, the surgical microscope has become an integral part of an operating room. The surgical microscope is typically a ceiling-mounted device that may be raised or lowered and positioned over any part of a patient's body. The surgical microscope often has multiple eyepieces that permit the surgeon and others to simultaneously view the magnified area under the microscope's objective lens.

A microscope drape, used to create a sterile barrier, may be affixed to the microscope at the lens housing of the objective lens to orient the drape with respect to the remaining structure of the microscope. Other portions of the drape may be spread and positioned to cover the remainder of the microscope structure. The objective lens housing for comparable surgical microscopes of different manufacturers may be of different sizes. Thus, a microscope drape that fits the objective lens barrel of one microscope may not fit the objective lens barrel of a similar microscope. Consequently, a larger and more expensive inventory of several different drapes is necessary to accommodate the different microscope objective lens barrels.

In addition, in order to be able to protect the objective lens and still be able to see the surgical area, a transparent protective lens is usually associated with the device that couples the drape to the microscope. The transparent protective lens, depending on its positioning, may cause an undesirable glare to the user of the microscope, which may complicate the surgical procedure.

### SUMMARY OF THE INVENTION

According to one embodiment of the invention, a microscope drape coupling system includes a first adapter configured to couple to a first objective lens barrel of a first microscope, a housing configured to rotatably couple to the first adapter, and a substantially flat transparent protective lens coupled within the housing such that a geometric normal to the transparent protective lens forms an angle with respect to an optical axis of a first objective lens housed within the first objective lens barrel when the housing is coupled to the first objective lens barrel.

Embodiments of the invention provide a number of technical advantages. Embodiments of the invention may include all, some, or none of these advantages. A microscope drape coupler, according to one embodiment, is adaptable to many different objective lens housing sizes, which reduces the number of drape couplers needed in inventory. The drape coupler may also include an angled transparent lens to substantially reduce or eliminate any glare that a user may encounter during surgery of other medical procedure. This angled transparent cover may be housed within a housing that rotates, which further may enhance its glare reduction capabilities. The housing is selectively removable from the drape coupler in case it needs to be changed out during a surgical procedure.

Other technical advantages are readily apparent to one skilled in the art from the following figures, descriptions, and claims.

### BRIEF DESCRIPTION OF THE DRAWINGS

For a more complete understanding of the invention, and for further features and advantages, reference is now made to the following description, taken in conjunction with the accompanying drawings, in which:

FIG. 1 is a perspective view of a microscope drape coupled to a microscope with a drape coupler in accordance with one embodiment of the present invention;

FIG. 2 is an exploded, perspective view of a microscope drape coupler in accordance with one embodiment of the present invention;

FIG. 3 is a cross-sectional view illustrating the microscope drape coupler of FIG. 2 coupled to a microscope in accordance with one embodiment of the present invention; and

FIG. 4 is a cross-sectional view illustrating the microscope drape coupler of FIG. 2 coupled to a microscope in accordance with another embodiment of the present invention.

### DETAILED DESCRIPTION OF EXAMPLE EMBODIMENTS OF THE INVENTION

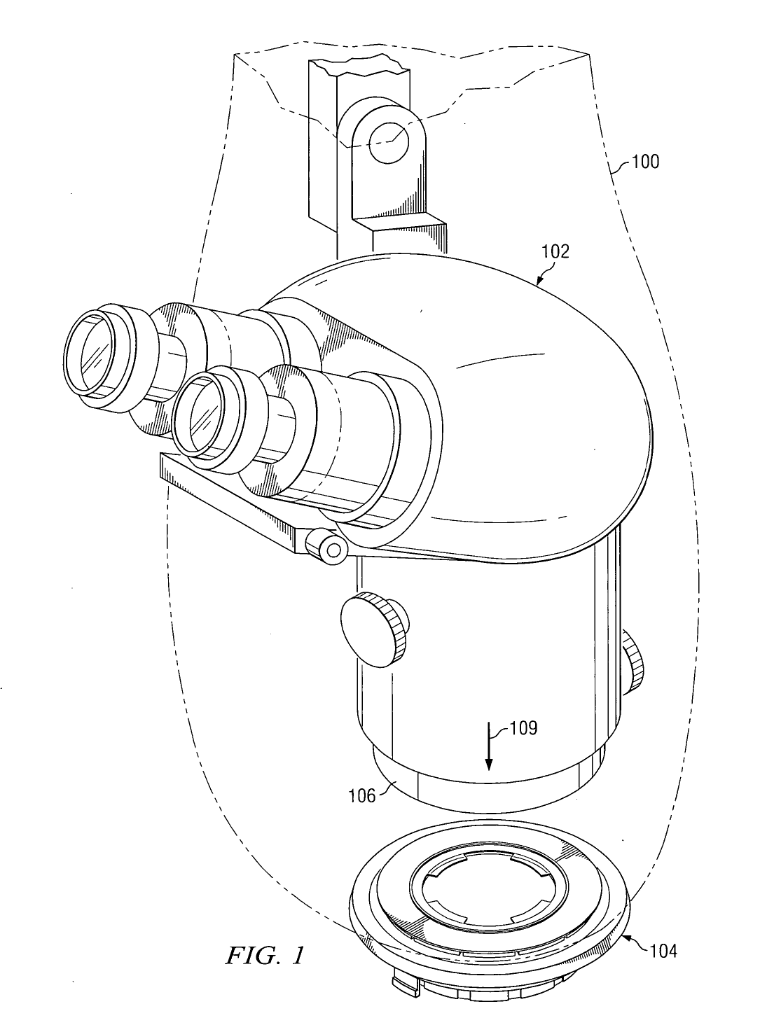

Example embodiments of the present invention and their advantages are best understood by referring now to FIGS. 1 through 4 of the drawings, in which like numerals refer to like parts.

FIG. 1 is a perspective view of a microscope drape 100 coupled to a microscope 102 with a drape coupler 104. Although the present invention contemplates any suitable medical instrument being protected by drape 100, the present invention is particularly suitable for surgical microscopes, such as microscope 102 illustrated in FIG. 1.

Drape 100 functions to generate a protective barrier between microscope 102 and its environment. For example, in an embodiment where microscope 102 is a surgical microscope, drape 100 protects microscope 102 from any bodily fluids, surgical fluids, and/or other materials during a surgical procedure from coming into contact with microscope 102. Conversely, any contaminants associated with microscope 102 are prevented from coming into contact with a patient during a surgical procedure. Any suitable drape 100 formed from any suitable material is contemplated by the present invention to cover microscope 102.

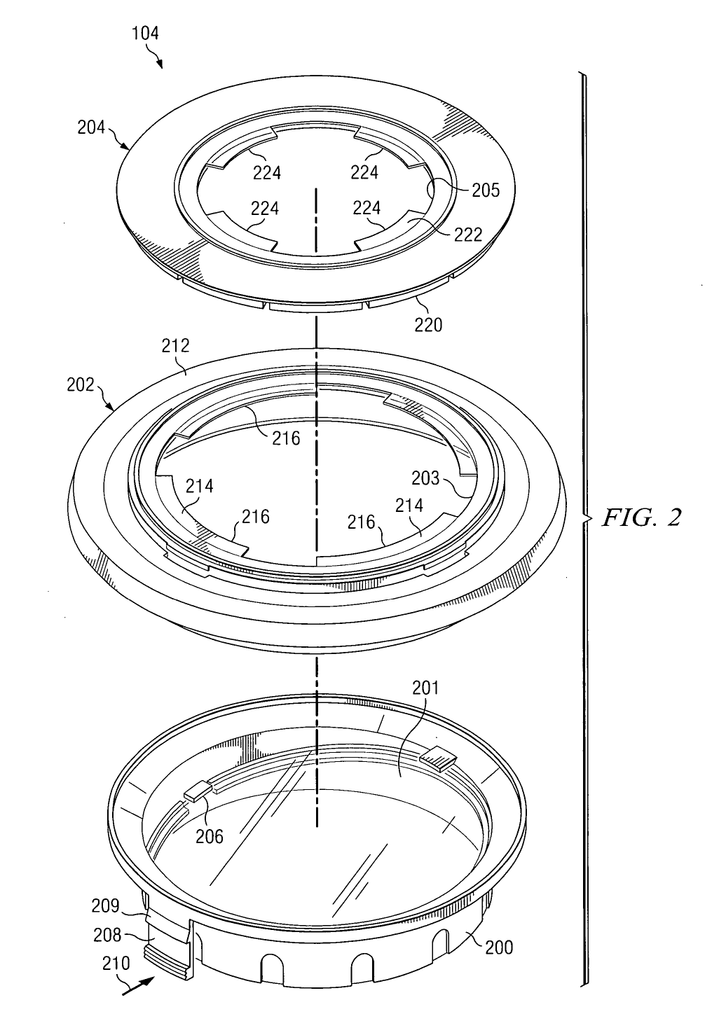

In the illustrated embodiment, drape coupler 104 couples to an objective lens barrel 106 of microscope 102; however, drape coupler 104 may couple to other portions of microscope 102 within the teachings of the present invention. Because there are many different types of microscopes available in the medical industry, many different sizes of objective lens barrels are encountered. Thus, a microscope drape coupler that fits the objective lens barrel of one microscope may not fit the objective lens barrel of another microscope. In addition, in order to be able to protect the objective lens of a microscope and still be able to see a surgical area, a transparent protective lens is typically associated with the drape coupler. The transparent protective lens, depending on its positioning, may cause an undesirable glare to the user of the microscope, which may complicate the surgical procedure. This transparent protective lens may also encounter bodily and/or surgical fluids during a surgical procedure, which means that the lens may need to be replaced during the surgical procedure. The present invention addresses these problems, and others, by providing drape coupler 104, as described in more detail below in conjunction with FIG. 2.

FIG. 2 is an exploded, perspective view of microscope drape coupler 104 according to one embodiment of the present invention. An advantage of microscope drape coupler 104 is that it is adaptable to many different sizes of objective lens barrels and includes an angled transparent lens housed within a rotatable housing to substantially reduce or eliminate any glare that a user may encounter during a surgical procedure. Hence, in the illustrated embodiment, drape coupler 104 includes a housing 200 having a transparent protective lens 201 coupled thereto, a first adapter 202 having an objective lens barrel aperture 203, and a second adapter 204 having an objective lens barrel aperture 205. As described in more detail below, second adapter 204 may not be needed in some embodiments depending on the size of objective lens barrel 106 of microscope 102 (FIG. 1).

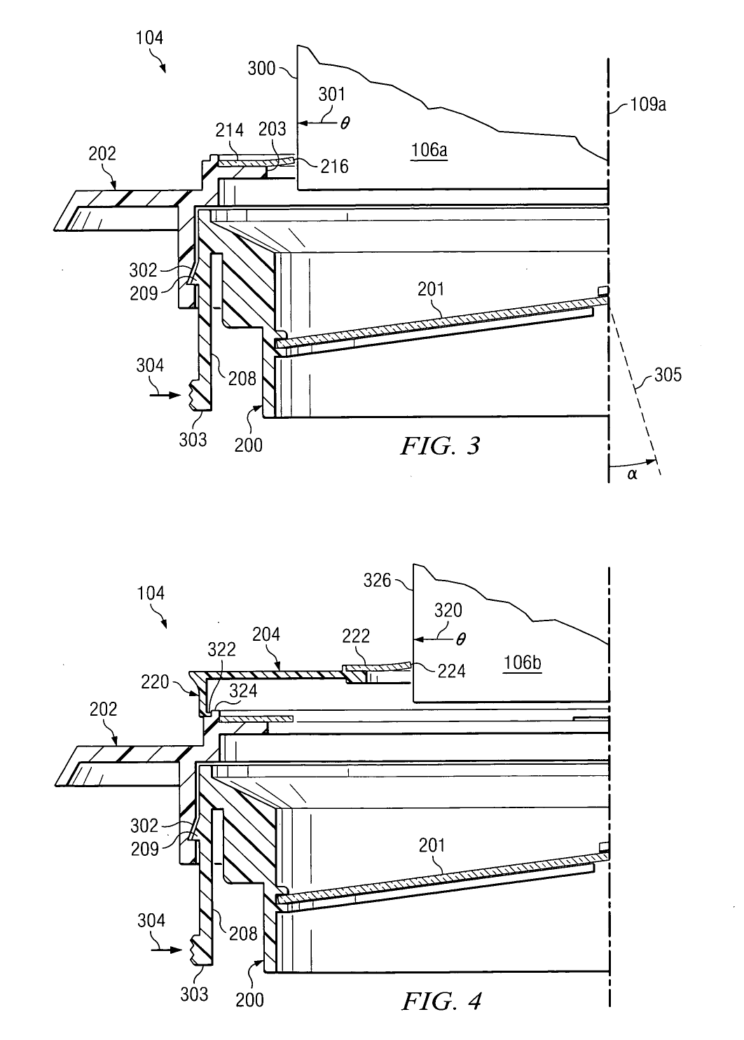

Housing 200 may be any suitable size and shape and may be formed from any suitable material. Housing 200 functions to house transparent protective lens 201. As described in more detail below in conjunction with FIGS. 3 and 4, housing 200 is rotatably coupled to first adapter 202 in order to facilitate the rotation of transparent protective lens 201 to substantially reduce or eliminate any glare during a surgical procedure. In one embodiment, housing 200 is removably coupled to first adapter 202 in order to facilitate the removal of housing 200 during a surgical procedure for replacing a transparent protective lens 201 in case lens 201 becomes damaged or unusable during a surgical procedure. Housing 200 may be rotatably and/or removably coupled to first adapter 202 in any suitable manner. In the illustrated embodiment, housing 200 includes a pair of locking tabs 208 disposed around a perimeter thereof. Each locking tip 208 includes a tongue 209 that is configured to engage a respective groove formed on first adapter 202, as described in greater detail below in conjunction with FIGS. 3 and 4. Any suitable number of locking tabs 208 may be disposed around the perimeter of housing 200 to facilitate the coupling of housing 200 to first adapter 202.

Transparent protective lens 201 may be formed from any suitable transparent material. Lens 201 may be coupled within housing 200 in any suitable manner. In one embodiment, a plurality of tabs 206 are utilized to secure lens 201 therein. As described above, lens 201 is angled within housing 200 such that when housing 200 is coupled to objective lens barrel 106 (FIG. 1), a geometric normal to transparent protective lens 201 forms an angle with respect to an optical axis 109 (FIG. 1) of an objective lens disposed within objective lens barrel 106. Any suitable angle may be utilized for lens 201. This angling of lens 201 facilitates the substantial reduction or elimination of any glare encountered during a surgical procedure. Depending on the lighting within an operating room, a glare may occur to a user of microscope 102. In order to reduce or eliminate that glare, the user merely rotates housing 200 to change the angle of refraction of the light so that it does not shine into the objective lens of microscope 102 in an undesirable manner.

First adapter 202 functions to couple housing 200 to objective lens barrel 106 of microscope 102 (FIG. 1). First adapter 202 also couples to drape 100 via an annular surface 212 disposed around a perimeter of first adapter 202. Drape 212 may be coupled to annular surface 212 in any suitable manner, such as adhesive coupling. The coupling of housing 200 to first adapter 202 is described in further detail below in conjunction with FIGS. 3 and 4.

First adapter 202 includes objective lens barrel aperture 203 and may have any suitable size and shape. Although a generally circular shape of objective lens barrel aperture 203 is illustrated in FIG. 2, other suitable shapes are contemplated by the present invention. Objective lens barrel aperture 203 has a diameter slightly larger than the diameter of objective lens barrel 106 of microscope 102. In order to couple to objective lens barrel 106, objective lens barrel aperture 203 includes a mounting ring 214 coupled thereto. Mounting ring 214 includes a mounting aperture 216 that has a diameter slightly smaller than the diameter of objective lens barrel 106 such that when a user positions first adapter 202 over objective lens barrel 106 mounting ring 214 deflects in such a manner that mounting aperture 216 elastically constricts about objective lens barrel 106 and holds first adapter 202 in place. In one embodiment, mounting ring 214 is formed from a flexible material, such as an elastomer, in order to facilitate the elastic constriction of mounting ring 214 on the outside surface of objective lens barrel 106.

In the illustrated embodiment, mounting ring 214 includes a plurality of tabs disposed around the circumference of objective lens barrel aperture 203. Any suitable number of tabs may be utilized. The use of tabs makes it easier for first adapter 202 to couple to objective lens barrels having slightly different diameters. For example, if the diameters of two different objective lens barrels are only a few millimeters different, then the tabs reduce the effort required to position first adapter 202 over the slightly larger objective lens barrel. Instead of tabs, the present invention contemplates mounting ring 214 being a continuous annular element having any suitable width.

Second adapter 204 functions to couple housing 200 and first adapter 202 to an objective lens barrel that has a diameter smaller than mounting aperture 216 of first aperture 202. As described above, second adapter 204 includes objective lens barrel aperture 205 that may have any suitable size and shape. Although a generally circular shape of objective lens barrel aperture 205 is illustrated in FIG. 2, other suitable shapes are contemplated by the present invention. Objective lens barrel aperture 205 has a diameter slightly larger than the diameter of an objective lens barrel to which it attaches (not illustrated).

Coupled to objective lens barrel aperture 205 is a mounting ring 222 similar to mounting ring 214 of first adapter 202, except that mounting ring 222 has a smaller diameter. Mounting ring 222 includes a mounting aperture 224 that has a diameter slightly smaller than the diameter of the objective lens barrel to which it attaches in order to facilitate the coupling of second adapter 204 thereto. Similar to mounting ring 214, mounting ring 222 may be a continuous annular ring coupled to objective lens barrel aperture 205 or may include a plurality of tabs, as illustrated in FIG. 2. In addition, mounting ring 224 may be formed from any suitable material that facilitates the elastic constriction of mounting ring 224 about an outside surface of an objective lens barrel to which it attaches.

In the illustrated embodiment, second adapter 204 includes a plurality of protrusions 220 to facilitate the coupling of second adapter 204 to first adapter 202. As described in more detail below in conjunction with FIG. 4, protrusions 220 also allow first adapter 202 to be rotatably coupled to second adapter 204. Additional details on the use of second adapter 204 are described below in conjunction with FIG. 4.

Thus, in one embodiment, drape coupler 104 is a universally adaptable drape coupler that functions to couple a drape to many different sizes of objective lens barrels of different microscopes in addition to having a rotatable and removable housing that houses a transparent protective lens that is angled with respect to the optical axis of the optical lens housed within the objective lens barrel to which it attaches. Having first adapter 202 and second adapter 204 reduces the number of drape couplers needed in inventory, which reduces cost. The interaction between housing 200, first adapter 202, and second adapter 204 is illustrated below in conjunction with FIGS. 3 and 4. FIG. 3 illustrates an embodiment where second adapter 204 is not utilized and FIG. 4 illustrates an embodiment where second adapter 204 is utilized because of a smaller diameter objective lens barrel.

Referring to FIG. 3, drape coupler 104 is illustrated as being coupled to an objective lens barrel 106a. As shown in FIG. 3, mounting ring 214 is illustrated as elastically constricting about an outside surface 300 of objective lens barrel 106a. This is because the diameter of mounting aperture 216 is slightly smaller than a diameter 301 of objective lens barrel 106a. To couple first adapter 202 to objective lens barrel 106a, a user simply places first adapter 202 over the outside surface 300 of objective lens barrel 106a in order to deflect mounting ring 214 so that it may “grip” the outside surface 300 and hold drape coupler 104 in place. Helping to keep drape coupler 104 in place is drape 100 (not shown in FIG. 3) which is pulled up and around microscope 102 and fastened in any suitable manner to a top portion of microscope 102.

Also illustrated in FIG. 3 is the coupling of housing 200 to first adapter 202. As described above, housing 202 includes one or more locking tabs 208 on outsider perimeter thereof. Each locking tab 208 has a tongue 209 that fits within a groove 302 formed on an inside surface of first adapter 202. Both tongue 209 and groove 302 may have any suitable profile that facilitates the coupling of housing 200 to first adapter 202 in addition to facilitating the rotation of housing 202 within the first adapter 202. In order to engage tongue 209 and groove 302, locking tab 208 includes a suitable resiliency in order for a user to apply a force to an end 303 of locking tab 208 as denoted by arrow 304. In other words, if two tabs are utilized, such as shown in FIG. 2, a user simply pushes ends 303 of each tab inwardly so that housing 202 may be inserted within first adapter 202 in order to engage tongues 209 and groove 302. To remove housing 200 from first adapter 202 the user simply reverse this process. Also illustrated in FIG. 3 is angled lens 201 that has a geometric normal 305 that forms an angle alpha with respect to an optical axis 109a of an objective lens housed within objective lens barrel 106a.

Referring now to FIG. 4, drape coupler 104 is illustrated as being coupled to an objective lens barrel 106b. In this embodiment, a diameter 320 of objective lens barrel 106b is smaller than mounting aperture 216 of mounting ring 214 of first aperture 202. Therefore, second adapter 204 is needed in this embodiment in order to couple drape coupler 104 to objective lens barrel 106b. Thus, the interaction of housing 200 and first adapter 202 in FIG. 4 is similar to the interaction of those two elements in FIG. 3; however, second adapter 204 is coupled to first adapter 202 in order to couple drape coupler 104 to objective lens barrel 106b. This is facilitated by protrusions 220 that each have a finger 322 projecting radially inwardly therefrom in order to grip an outside surface 324 of first adapter 202. In this manner, a user simply press fits second adapter 204 over first adapter 202 such that fingers 322 grip outside surface 324 and hold first adapter 202 in place, as well as allowing first adapter 202 to be rotatable with respect to second adapter 204. Also helping to keep drape coupler 104 in place is drape 100 (not shown in FIG. 4), which is pulled up and around microscope 102 and fastened in any suitable manner to a top portion of microscope 102.

As illustrated in FIG. 4, mounting ring 222 is shown in a slightly deflected position because mounting aperture 224 has a diameter slightly smaller than a diameter 320 of objective lens barrel 106b. Therefore, a user simply places second adapter 204 over an outside surface 326 of objective lens barrel 106b in order to elastically constrict mounting ring 222 about surface 326 in order to couple second adapter 204 thereto.

Although embodiments of the invention and some of their advantages are described in detail, a person skilled in the art could make various alterations, additions, and omissions without departing from the spirit and scope of the present invention as defined by the appended claims.

What is claimed is:

1. A microscope drape coupling system, comprising:
a first adapter configured to couple to a first objective lens barrel of a first microscope, the first adapter comprising:
an annular surface disposed around a perimeter of the first adapter and adapted to couple to a drape; and
a mounting ring defining a mounting aperture adapted to couple to the first objective lens barrel, the diameter of the mounting aperture slightly smaller than the diameter of the objective lens barrel;
a housing configured to rotatably couple to the first adapter; and
a substantially flat transparent protective lens within the housing such that a geometric normal to the transparent protective lens forms an angle with respect to an optical axis of a first objective lens housed within the first objective lens barrel when the housing is coupled to the first objective lens barrel.

2. A microscope drape coupling system, comprising:
a first adapter configured to couple to a first objective lens barrel of a first microscope;
a housing configured to rotatably couple to the first adapter; and
a substantially flat transparent protective lens within the housing such that a geometric normal to the transparent protective lens forms an angle with respect to an optical axis of a first objective lens housed within the first objective lens barrel when the housing is coupled to the first objective lens barrel.

3. The microscope drape coupling system of claim 2, wherein the housing is selectively removable from the first adapter via one or more locking tabs disposed around a perimeter of the housing.

4. The microscope drape coupling system of claim 2, further comprising a second adapter configured to rotatably couple to the first adapter and further configured to couple to a second objective lens barrel of a second microscope.

5. The microscope drape coupling system of claim 2, wherein the first adapter further comprises an annular surface disposed around a perimeter thereof, the annular surface adapted to couple to a drape.

6. The microscope drape coupling system of claim 2, wherein the housing comprises one or more locking tabs disposed around a perimeter thereof, each locking tab comprising a tongue adapted to engage a respective groove formed on the first adapter for rotatably coupling the housing to the first adapter, and wherein the housing is selectively removable from the first adapter via the locking tabs.

7. A microscope drape coupling system, comprising:
a first adapter having a first objective lens barrel aperture;
a first mounting ring coupled to the first adapter and having a first mounting aperture configured to couple the first adapter to a first objective lens barrel of a first microscope;
a second adapter having a second objective lens barrel aperture;
a second mounting ring coupled to the second adapter and having a second mounting aperture configured to couple the second adapter to a second objective lens barrel of a second microscope, the second mounting aperture having a diameter smaller than the first mounting aperture;
a housing having a plurality of locking tabs disposed around a perimeter thereof, each locking tab comprising a tongue adapted to engage a groove formed on the first adapter for rotatably coupling the housing to the first adapter, the housing selectively removable from the first adapter via the locking tabs; and
a substantially flat transparent protective lens within the housing such that a geometric normal to the transparent protective lens forms an angle with respect to a longitudinal axis of either the first or second objective lens barrel when the housing is coupled to either the first or second objective lens barrel.

8. The microscope drape coupling system of claim 7, wherein the first and second mounting rings are each formed from a flexible material such that the first and second mounting rings are each adapted to elastically constrict about the first and second objective lens barrels, respectively.

9. The microscope drape coupling system of claim 7, wherein the first mounting ring comprises a plurality of tabs extending inwardly from the first objective lens barrel aperture, wherein the first mounting aperture is defined by the inside edges of each tab.

10. The microscope drape coupling system of claim 7, wherein the second mounting ring comprises a plurality of tabs extending inwardly from the second objective lens barrel aperture, wherein the second mounting aperture is defined by the inside edges of each tab.

11. The microscope drape coupling system of claim 7, wherein the second adapter comprises a plurality of protrusions configured to rotatably couple the second adapter to the first adapter.

12. A method of coupling a drape to a microscope, comprising:
providing a first adapter having a first objective lens barrel aperture and a first mounting ring, the first mounting ring having a first mounting aperture;
rotatably coupling a housing to the first adapter, the housing having a substantially flat transparent protective lens therein such that a geometric normal to the transparent protective lens forms an angle with respect to an optical axis of a first objective lens housed within a first objective lens barrel when the housing is coupled to the first objective lens barrel; and
coupling the first adapter to the first objective lens barrel, the first objective lens barrel having an outside diameter slightly larger than a diameter of the first mounting aperture and slightly smaller than a diameter of the first objective lens barrel aperture.

13. The method of claim 12, further comprising:
providing a second adapter having a second objective lens barrel aperture and a second mounting ring, the second mounting ring having a second mounting aperture;
coupling the second adapter to the first adapter; and
coupling the second adapter to a second objective lens barrel, the second objective lens barrel having an outside diameter slightly larger than a diameter of the second mounting aperture and slightly smaller than a diameter of the second objective lens barrel aperture.

14. The method of claim 13, further comprising coupling a drape to an annular surface disposed around a perimeter of the first adapter.

15. The method of claim 12, wherein coupling the first adapter to the first objective lens barrel comprises elastically constricting the first mounting ring about the first objective lens barrel.

16. The method of claim 13, wherein coupling the second adapter to the second objective lens barrel comprises elastically constricting the second mounting ring about the second objective lens barrel.

17. The method of claim 12, wherein the first mounting ring comprises a plurality of tabs extending inwardly from the first objective lens barrel aperture, wherein the first mounting aperture is defined by the inside edges of each tab.

18. The method of claim 13, wherein the second mounting ring comprises a plurality of tabs extending inwardly from the second objective lens barrel aperture, wherein the second mounting aperture is defined by the inside edges of each tab.

19. The method of claim 12, wherein rotatably coupling the housing to the first adapter comprises engaging a plurality of tongues on respective ones of a plurality of tabs formed on the housing with respective ones of a plurality of grooves formed on the first adapter.

20. A microscope drape coupling system, comprising:
a first adapter having a first objective lens barrel aperture;
a first mounting ring coupled to the first adapter and having a first mounting aperture configured to couple the first adapter to a first objective lens barrel of a first microscope that has an outside diameter slightly larger than a diameter of the first mounting aperture and slightly smaller than a diameter of the first objective lens barrel aperture;
a housing configured to rotatably couple to the first adapter; and
a substantially flat transparent protective lens within the housing such that a geometric normal to the transparent protective lens forms an angle with respect to an optical axis of a first objective lens housed within the first objective lens barrel when the housing is coupled to the first objective lens barrel.

21. The microscope drape coupling system of claim 20, further comprising a second adapter configured to couple to the first adapter, the second adapter comprising:
a second objective lens barrel aperture; and
a second mounting ring coupled to the second adapter and having a second mounting aperture configured to couple the second adapter to a second objective lens barrel of a second microscope that has an outside diameter slightly larger than a diameter of the second mounting aperture and slightly smaller than a diameter of the second objective lens barrel aperture, the second mounting aperture having a diameter smaller than the first mounting aperture.

22. The microscope drape coupling system of claim 21, wherein the second adapter comprises a plurality of protrusions configured to rotatably couple the second adapter to the first adapter.

23. The microscope drape coupling system of claim 20, wherein the first adapter further comprises an annular surface disposed around a perimeter thereof, the annular surface adapted to couple to a drape.

24. The microscope drape coupling system of claim 20, wherein the first mounting ring is formed from a flexible material such that the first mounting ring is adapted to elastically constrict about the first objective lens barrel in order to couple the first adapter thereto.

25. The microscope drape coupling system of claim 21, wherein the second mounting ring is formed from a flexible material such that the second mounting ring is adapted to elastically constrict about the second objective lens barrel in order to couple the second adapter thereto.

26. The microscope drape coupling system of claim 20, wherein the first mounting ring comprises a plurality of tabs extending inwardly from the first objective lens barrel aperture, wherein the first mounting aperture is defined by the inside edges of each tab.

27. The microscope drape coupling system of claim 21, wherein the second mounting ring comprises a plurality of tabs extending inwardly from the second objective lens barrel aperture, wherein the second mounting aperture is defined by the inside edges of each tab.

28. The microscope drape coupling system of claim 20, wherein the housing comprises one or more locking tabs disposed around a perimeter thereof, each locking tab comprising a tongue adapted to engage a respective groove formed on the first adapter for rotatably coupling the housing to the first adapter, and wherein the housing is selectively removable from the first adapter via the locking tabs.
